(19) United States
(12) Patent Application Publication
(10) Pub. No.: US 2019/0365644 A1
(43) Pub. Date: Dec. 5, 2019

(54) DRUG DELIVERY SYSTEM

(71) Applicants: Aram Sahakian, Glendale, CA (US); Travis Turner, Beverly Hills, CA (US); Vicken Sahakian, Los Angeles, CA (US)

(72) Inventors: Aram Sahakian, Glendale, CA (US); Travis Turner, Beverly Hills, CA (US); Vicken Sahakian, Los Angeles, CA (US)

(21) Appl. No.: 16/540,919

(22) Filed: Aug. 14, 2019

Related U.S. Application Data
(62) Division of application No. 16/386,920, filed on Apr. 17, 2019.
(60) Provisional application No. 62/659,588, filed on Apr. 18, 2018.

(51) Int. Cl. A61K 9/00; B29C 48/00; A61K 31/353; B29C 45/14; A61K 47/14; B29C 48/21; A61K 31/065; A61K 47/30

(52) U.S. Cl. CPC A61K 9/0036; B29C 48/0022; A61K 31/353; B29C 45/14; B29L 2031/753; B29C 48/21; A61K 31/065; A61K 47/30; A61K 47/14

(57) ABSTRACT

A drug delivery system configured to be inserted transvaginally. The drug delivery system includes an outer skin defining a reservoir, and a mixture of a carrier oil and one or more cannabinoids inside the reservoir. The outer skin of the drug delivery device is permeable to the mixture of the carrier oil and the one or more cannabinoids. The drug delivery device may be a resilient ring, and the one or more cannabinoids may include Cannabidiol and less than approximately 0.03% Tetrahydrocannabinol.

### CROSS-REFERENCE TO RELATED APPLICATION(S)

This application is a divisional of U.S. patent application Ser. No. 16/386,920, filed Apr. 17, 2019, which claims priority to and the benefit of U.S. Provisional Application No. 62/659,588, filed Apr. 18, 2018, the entire contents of both of which are incorporated herein by reference.

### BACKGROUND

A variety of symptoms are commonly associated with the menstrual cycle, such as abdominal and pelvic cramping pains, breast tenderness, and mood changes. Conventional methods for treating these symptoms include oral ingestion of multiple analgesics for the entire duration of the menstrual cycle. However, these oral analgesics must be repeatedly taken over the course of the menstrual cycle and they may cause unwanted side effects.

### BRIEF DESCRIPTION OF THE DRAWINGS

The features and advantages of embodiments of the present disclosure will become more apparent by reference to the following detailed description when considered in conjunction with the following drawings. In the drawings, like reference numerals are used throughout the figures to reference like features and components. The figures are not necessarily drawn to scale.

FIGS. 1A-1B are a perspective view and a cross-sectional view, respectively, of a drug delivery device according to one embodiment of the present disclosure;

FIGS. 2A-2C are a front view and cross-sectional views, respectively, of a drug delivery device according to another embodiment of the present disclosure;

FIGS. 3A-3F illustrate an embodiment of the drug delivery device of the present disclosure being inserted transvaginally into a user; and

FIG. 4 illustrates tasks of a method of manufacturing a drug delivery device according to one embodiment.

### SUMMARY

The present disclosure is directed to various embodiments of a drug delivery device configured to be inserted transvaginally into a user. In one embodiment, the drug delivery device includes a core including a mixture of a carrier oil and one or more cannabinoids and a skin covering the core. The skin is permeable to the mixture of the carrier oil and the one or more cannabinoids.

The drug delivery device may be a resilient ring.

The one or more cannabinoids may include Cannabidiol and/or Tetrahydrocannabinol.

The one or more cannabinoids may include less than approximately 0.03% Tetrahydrocannabinol.

The carrier oil may include one or more medium chain triglycerides.

The skin may be substantially free of cannabinoids prior to transvaginal insertion.

The core may include a thermoplastic polymer or an elastomer material.

The one or more cannabinoids may be supersaturated in the thermoplastic polymer or the elastomer material of the core.

In another embodiment, the drug delivery device may include an outer skin defining a reservoir and a mixture of a carrier oil and one or more cannabinoids inside the reservoir. The outer skin is permeable to the mixture of the carrier oil and the one or more cannabinoids.

The drug delivery device may be a resilient ring.

The one or more cannabinoids may include Cannabidiol and/or Tetrahydrocannabinol.

The one or cannabinoids may include less than approximately 0.03% Tetrahydrocannabinol.

The carrier oil may include one or more medium chain triglycerides.

The outer skin may be substantially free of cannabinoids prior to transvaginal insertion.

The present disclosure is also directed to various methods of treating symptoms associated with a menstrual cycle. In one embodiment, the method includes inserting a drug delivery device transvaginally into a user. The drug delivery device includes a mixture of a carrier oil and one or more cannabinoids and a membrane covering the mixture. The membrane is permeable to the mixture.

The present disclosure is also directed to various methods of manufacturing a drug delivery device. In one embodiment, the method includes refining one or more cannabinoids into a crystalline resin, and mixing the crystalline resin with a carrier oil to form a mixture.

The method may also include inserting the mixture of the carrier oil and the one or more cannabinoids into a core, forming a skin covering the core, and shaping the core and the skin into a desired shape of the drug delivery device.

Shaping the core and the skin into the desired shape may include co-extruding the core and the skin to form an extruded core and skin, cutting the extruded core and skin into segments having a desired length, and connecting ends of each individual segment together to form the drug delivery device.

The method may include forming an outer skin, shaping the outer skin into a desired shape, and injecting the mixture of the carrier oil and one or more cannabinoids into a reservoir defined by the outer skin.

Refining the one or more cannabinoids may include extracting live resin from at least one of a cannabis sativa plant and an indica plant, and refining the live resin extracted from the cannabis sativa plant and/or the indica plant into the crystalline resin.

This summary is provided to introduce a selection of concepts that are further described below in the detailed description. This summary is not intended to identify key or essential features of the claimed subject matter, nor is it intended to be used in limiting the scope of the claimed subject matter. One or more of the described features may be combined with one or more other described features to provide a workable device.

### DETAILED DESCRIPTION

The present disclosure is directed to various embodiments of a drug delivery device for vaginal administration of a therapeutically effective amount of a pharmaceutical grade cannabinoid formulation. The drug delivery system of the present disclosure is configured to locally administer one or more cannabinoids directly to the vaginal fossa and/or the vaginal mucosa of the user. The one or more cannabinoids that are administered to the vaginal fossa and/or the vaginal mucosa are transmitted to local perineal and uterine pain receptors (e.g., the direct exposure of the vaginal mucosa to the one or more cannabinoids results in the rapid absorption of these one or more cannabinoids into adjacent tissue and organs). In this manner, the drug delivery device of the present disclosure is configured to alleviate pain and discomfort caused by uterine contractility and uterine blood flow associated with the menstrual cycle, such as abdominal and pelvic cramping pains. Additionally, the drug delivery device of the present disclosure is configured to supply a sustained delivery of the one or more cannabinoids during the entire menstrual cycle, such as from 5 to 10 days. The drug delivery system of the present disclosure may also be utilized to provide relief of premenstrual symptomatology, such as breast tenderness and/or mood changes (e.g., irritability).

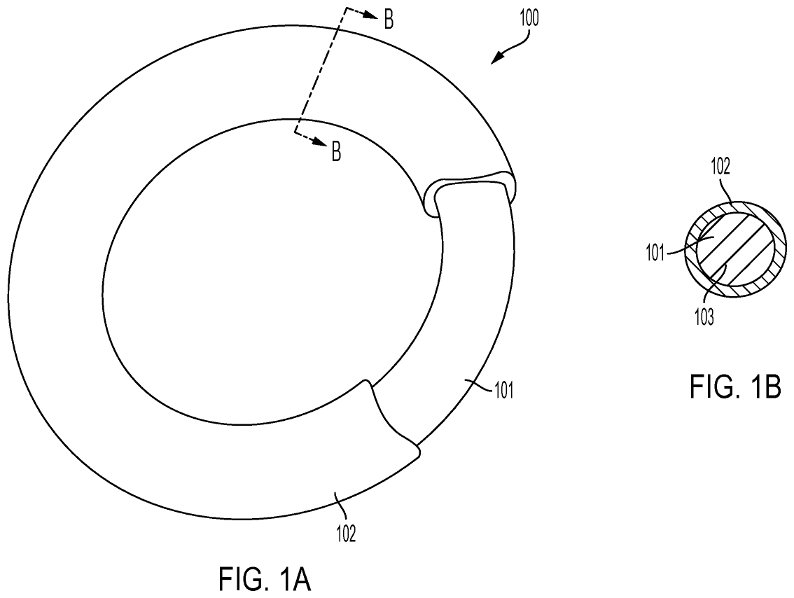

With reference now to FIGS. 1A-1B, an intravaginal drug delivery device 100 according to one embodiment of the present disclosure includes a core 101 and a skin 102 (e.g., a membrane) covering the core 101. In one or more embodiments, the skin 102 may completely or substantially completely surround an exterior surface of the core 101. In FIG. 1A, a portion of the skin 102 is broken away to reveal the underlying core 101. The core 101 includes a mixture 103 of a carrier oil (e.g., medium chain triglycerides (MCT oil)) and one or more cannabinoids, such as Cannabidiol (CBD) and/or Tetrandrocannabinol (THC). In one or more embodiments, the one or more cannabinoids in the core 101 of the drug delivery device 100 have a THC content less than approximately 0.03% (e.g., less than approximately 0.01%) such that the one or more cannabinoids are not psychoactive or are substantially not psychoactive. In one or more embodiments, the skin 102 is not provided with the one or more cannabinoids (e.g., the drug delivery device 100 includes a drug-loaded core 101 and a non-medicated outer skin 102). That is, in one or more embodiments, prior to transvaginal implantation of the drug delivery device 100, the skin 102 is free or substantially free of cannabinoids.

The skin 102 is configured to permit diffusion of the mixture 103 of the carrier oil and the one or more cannabinoids from the core 101 into (e.g., through) the skin 102 such that the mixture 103 can be delivered to a user (e.g., administered vaginally). In the illustrated embodiment, the skin 102 defines the exterior of the drug delivery device 100 such that when the drug delivery device 100 is inserted transvaginally into a user and the mixture 103 of the carrier oil and the one or more cannabinoids diffuse from the core 101 into or through the skin 102, the one or more cannabinoids are in directly administered to the vaginal fossa and/or the vaginal mucosa of the user.

In one or more embodiments, the core 101 may include any thermoplastic polymer or elastomer material suitable for pharmaceutical use, such as ethylene-vinylacetate copolymer (poly-EVA), low density polyethylene, or styrene-butadiene-styrene copolymer. In one or more embodiments in which the core 101 includes poly-EVA, the core 101 may have a vinyl acetate content in a range from approximately 25% to approximately 35% (e.g., in a range from approximately 26% to approximately 30%).

In the illustrated embodiment, the drug delivery device 100 is in the form of a ring configured for vaginal insertion (e.g., the core 101 is a solid annular member and the skin 102 is a hollow annular member around the core 101). The ring may have any size suitable for vaginal insertion, such as, for instance, an outer diameter in a range from approximately 50 mm and approximately 60 mm (e.g., in a range from approximately 52 mm and approximately 56 mm). Additionally, in one or more embodiments, a cross-sectional diameter of the ring may be in a range from approximately 2.5 mm and approximately 5 mm. Although in the illustrated embodiment the ring is annular, in one or more embodiments, the ring may have any other shape suitable for transvaginal insertion, such as, for instance, a squared ring shape, an ellipse, or an oval. Additionally, in one or more embodiments, the core 101 and/or the skin 102 may include one or more features that increase the surface area of drug delivery device 100. Increasing the surface area of the drug delivery device 100 increases the rate at which the one or more cannabinoids are delivered compared to a drug delivery device without these one or more features. For instance, in one or more embodiments, the core 101 and the skin 102 may each have a wavy or undulating outer profile.

In one or more embodiments, a surface area of the core 101 may be greater than approximately 800 mm² or greater than approximately 1000 mm². In one or more embodiments, the surface area of the core 101 may be in a range from approximately 1700 mm² to approximately 2000 mm². In one or more embodiments, the surface area of the core 101 may be greater than 2000 mm².

In one or more embodiments, the one or more cannabinoids are dissolved in the thermoplastic polymer or the elastomer material (e.g., poly-EVA) of the core 101 up to a relatively low degree of supersaturation, such as, for instance, in a range from approximately 1 to approximately 6 times the saturation concentration of the one or more cannabinoids in the polymer at 25° C. (e.g., the one or more cannabinoids may be provided in an amount that is in a range of approximately 1-6 times the amount that is necessary to obtain a saturation concentration of the one or more cannabinoids in the polymer material of the core 101 at 25° C.). In one or more embodiments, the one or more cannabinoids may be dissolved in the thermoplastic polymer or the elastomer material of the core 101 at a concentration in a range from approximately 1 to approximately 5 times the saturation concentration of the one or more cannabinoids in the polymer. In one or more embodiments, the drug delivery device 100 is configured to release, on average, the one or more cannabinoids from the core 101 at a release rate in a range from approximately 15 mg to approximately 45 mg per 24 hours in situ. Additionally, in one or more embodiments, the drug delivery device 100 is configured to release the one or more cannabinoids from the core 101 over a period in a range from approximately 5 days to approximately 10 days. In one or more embodiments, the core 101 includes an amount of the one or more cannabinoids in a range from approximately 75 mg to approximately 450 mg. The amount of the one or more cannabinoids provided in the core 101 and the degree of supersaturation of the one or more cannabinoids in the thermoplastic polymer or the elastomer material of the core 101 may be selected depending on the desired release rate of the one or more cannabinoids and the desired release period of the one or more cannabinoids. Additionally, the carrier oil and the one or more cannabinoids (e.g., Cannabidiol and/or Tetrahydrocannabinol) may be provided in any suitable ratio in the core 101.

In one or more embodiments, the skin 102 of the drug delivery device 100 may include any thermoplastic polymer or elastomer material suitable for pharmaceutical use, such as ethylene-vinylacetate copolymer (poly-EVA), low density polyethylene, or styrene-butadiene-styrene copolymer. In one or more embodiments, the skin 102 and the core 101 of the drug delivery device 100 may be made out of the same material (e.g., poly-EVA). In one or more embodiments, the core 101 and/or the skin 102 may include one or more polysiloxanes. In one or more embodiments, the skin 102 and the core 101 of the drug delivery device 100 may be made out of different materials.

In one or more embodiments in which the skin 102 of the drug delivery device 100 includes poly-EVA, the skin 102 may have a thickness in a range from approximately 40 μm to approximately 300 μm and a vinyl acetate content in a range from approximately 5% to approximately 15%. In one or more embodiments in which the skin 102 of the drug delivery device 100 includes poly-EVA, the skin 102 may have a thickness of approximately 100 μm and a vinyl acetate content in a range from approximately 9% to approximately 10%. The various properties of the skin 102 (e.g., material and thickness) may be selected depending on the desired drug delivery rate (e.g., the various properties of the skin 102 and/or the concentration of one or more cannabinoids in the mixture 103 in the core 101 are selected to deliver a therapeutically effective amount of the one or more cannabinoids over the life of the drug delivery device 100).

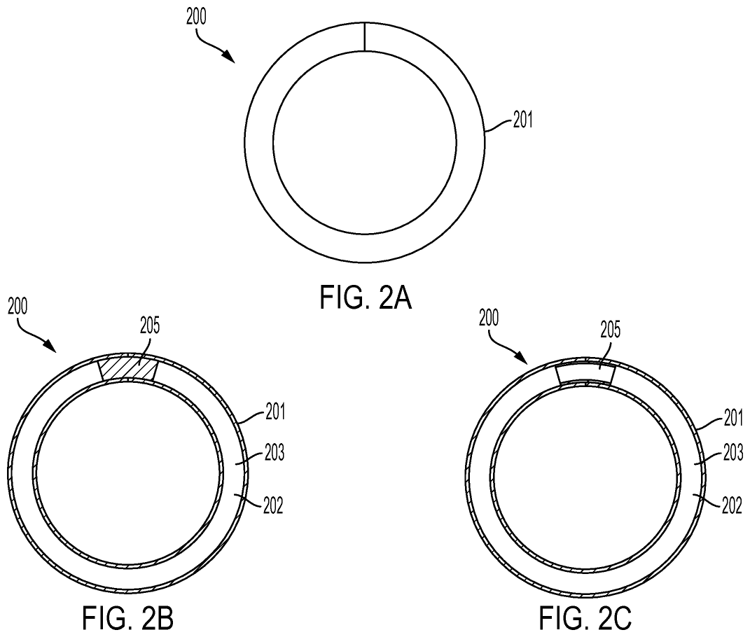

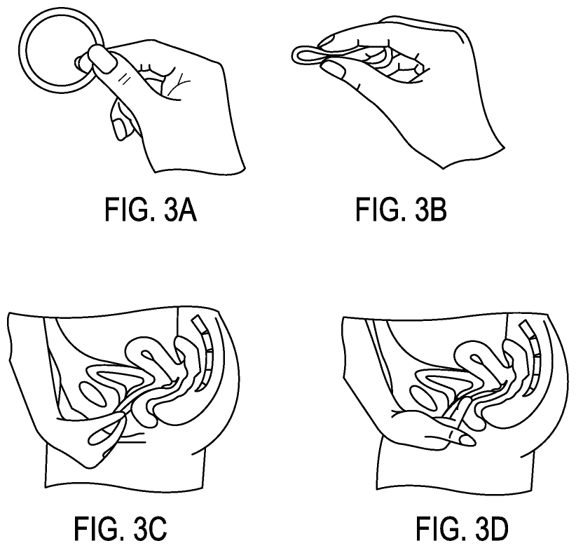

FIGS. 2A-2C illustrate an intravaginal drug delivery device 200 according to another embodiment of the present disclosure. In the illustrated embodiment, the intravaginal drug delivery device 200 includes an outer skin or membrane 201. In the illustrated embodiment, the outer skin 201 is a thin-walled, hollow member defining a reservoir or cavity 202 in an interior of the outer skin 201. The intravaginal drug delivery device 200 also includes a mixture 203 of a carrier oil (e.g., MCT oil) and one or more cannabinoids, such as Cannabidiol (CBD) and/or Tetrahydrocannabinol (THC), in the reservoir 202. In one or more embodiments, the mixture 203 fills or substantially fills the reservoir 202. In one or more embodiments, the mixture 203 may be the same as or similar to the mixture 103 described above with reference to FIGS. 1A-1B (e.g., the mixture 203 may have the same chemical composition, amount, and concentration as the mixture 103).

The outer skin 201 is configured to permit diffusion of the mixture 203 of the carrier oil and the one or more cannabinoids from the reservoir 202 into (e.g., through) the outer skin 201 such that the mixture 203 can be delivered to a user (e.g., administered vaginally). In the illustrated embodiment, the outer skin 201 defines the exterior of the drug delivery device 200 such that when the drug delivery device 200 is inserted transvaginally into a user and the mixture 203 of the carrier oil and the one or more cannabinoids diffuse from the reservoir 202 into or through the outer skin 201, the one or more cannabinoids are in directly administered to the vaginal fossa and/or the vaginal mucosa of the user.

The volume of the reservoir 202, the concentration of the mixture 203 in the reservoir 202, and the properties of the outer skin 201 may be selected to deliver a therapeutically effective amount of the one or more cannabinoids over the life of the drug delivery system 200. In one or more embodiments, the drug delivery system 200 is configured to release, on average, the one or more cannabinoids from the reservoir 202 at a release rate in a range from approximately 15 mg to approximately 45 mg per 24 hours in situ. Additionally, in one or more embodiments, the drug delivery system 200 is configured to release the one or more cannabinoids from the reservoir 202 over a period in a range from approximately 5 days to approximately 10 days. In one or more embodiments, the reservoir 202 includes an amount of the one or more cannabinoids in a range from approximately 75 mg to approximately 450 mg. The amount of the one or more cannabinoids provided in the reservoir 202 may be selected depending on the desired release rate of the one or more cannabinoids and the desired release period of the one or more cannabinoids. Additionally, the carrier oil and the one or more cannabinoids (e.g., Cannabidiol and/or Tetrahydrocannabinol) may be provided in any suitable ratio in the reservoir 202.

In one or more embodiments, the outer skin 201 may be formed of a thermoplastic polymer or a microporous polymer. In one or more embodiments, the outer skin 201 of the drug delivery device 200 may include any thermoplastic polymer or elastomer material suitable for pharmaceutical use, such as ethylene-vinylacetate copolymer (poly-EVA), low density polyethylene, or styrene-butadiene-styrene copolymer. In one or more embodiments, the outer skin 201 may include one or more polysiloxanes.

In one or more embodiments in which the outer skin 201 of the drug delivery device 200 includes poly-EVA, the outer skin 201 may have a thickness in a range from approximately 40 μm to approximately 300 μm and a vinyl acetate content in a range from approximately 5% to approximately 15%. In one or more embodiments in which the outer skin 201 of the drug delivery device 200 includes poly-EVA, the outer skin 201 may have a thickness of approximately 100 μm and a vinyl acetate content in a range from approximately 9% to approximately 10%. The various properties of the outer skin 201 (e.g., material and thickness) may be selected depending on the desired drug delivery rate (e.g., the various properties of the outer skin 201 and/or the concentration of the one or more cannabinoids in the mixture 203 in the reservoir 202 are selected to deliver a therapeutically effective amount of the one or more cannabinoids over the life of the drug delivery device 200).

Additionally, in the illustrated embodiment, the intravaginal drug delivery device 200 also includes a coupling member 204 coupling opposite ends of the outer skin 202 together such that the outer skin 202 forms an annular member (e.g., a ring). The coupling member 204 may have any suitable configuration for joining the ends of the outer skin 202 together. In one or more embodiments, the coupling member 204 is configured to form a fluid-tight or substantially fluid-tight connection between the ends of the outer skin 202. In one embodiment, the coupling member 204 may be a solid plug, as illustrated in FIG. 2B. In another embodiment, the coupling member 204 may include a passageway that permits the mixture 203 to flow through the passageway, as illustrated in FIG. 2C.

In the illustrated embodiment, the drug delivery device 200 is in the form of a ring configured for vaginal insertion (e.g., the outer skin 202 is a hollow annular member). The ring may have any size suitable for vaginal insertion, such as, for instance, an outer diameter in a range from approximately 50 mm and approximately 60 mm (e.g., in a range from approximately 52 mm and approximately 56 mm). Additionally, in one or more embodiments, a cross-sectional diameter of the ring may be in a range from approximately 2.5 mm and approximately 5 mm. Although in the illustrated embodiment the ring is annular, in one or more embodiments, the ring may have any other shape suitable for transvaginal insertion, such as, for instance, a squared ring shape, an ellipse, or an oval. Additionally, in one or more embodiments, the outer skin 202 may include one or more features that increase the surface area of drug delivery device 200. Increasing the surface area of the drug delivery device 200 increases the rate at which the one or more cannabinoids are delivered compared to a drug delivery device without these one or more features. For instance, in one or more embodiments, the outer skin 202 may have a wavy or undulating outer profile.

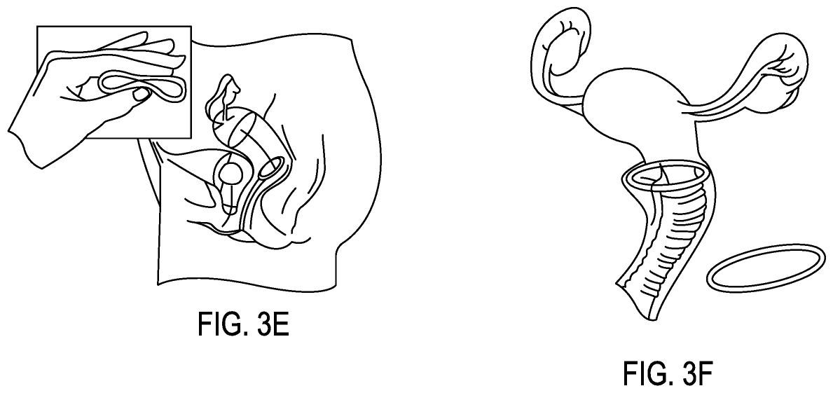

FIGS. 3A-3F illustrate a drug delivery device according to one embodiment of the present disclosure (e.g., the drug delivery device 100 illustrated in FIGS. 1A-1B or the drug delivery device 200 illustrated in FIGS. 2A-2C) being inserted transvaginally into a user. In one or more embodiments, the drug delivery system may be pinched to collapse the drug delivery system (e.g., collapse the ring) and then the collapsed drug delivery system may be inserted up the vaginal canal. Once the collapsed drug delivery system is in close proximity to the cervix and the uterus (e.g., once the drug delivery system is placed within approximately 3 cm or less of the cervix), the drug delivery system may be released. In the illustrated embodiment, the drug delivery system is resilient (e.g., the ring is resilient) such that the drug delivery system is configured to return to the uncollapsed configuration. As the drug delivery system returns to the uncollapsed configuration, the drug delivery system presses against the wall of the vaginal canal to retain the drug delivery system in therapeutically effective proximity to the cervix and the uterus. As described above, once the drug delivery system has been inserted into the vaginal cavity in close proximity to the cervix and the uterus, the drug delivery system of the present disclosure is configured to locally administer one or more cannabinoids directly to the vaginal fossa and/or the vaginal mucosa. The one or more cannabinoids that are administered to the vaginal fossa and/or the vaginal mucosa are transmitted to local perineal and uterine pain receptors (e.g., the direct exposure of the vaginal mucosa to the one or more cannabinoids results in the rapid absorption of these one or more cannabinoids into adjacent tissue and organs). In this manner, the drug delivery system of the present disclosure is configured to alleviate pain and discomfort caused by uterine contractility and uterine blood flow associated with the menstrual cycle, such as abdominal and pelvic cramping pains.

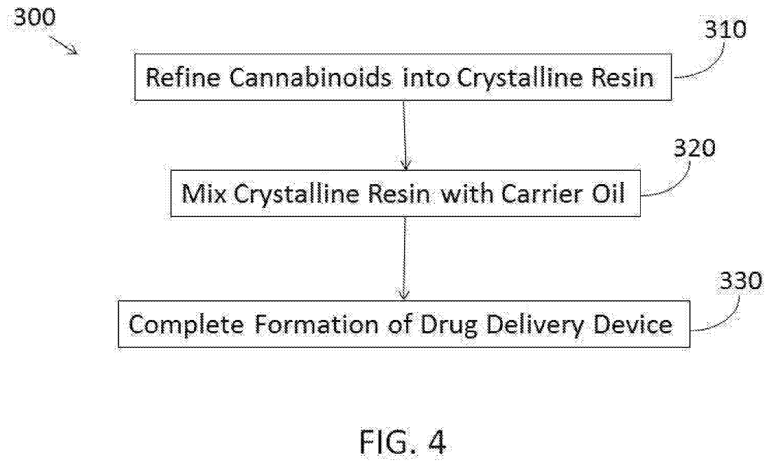

The present disclosure is also directed to various methods of manufacturing a drug delivery device of the present disclosure (e.g., the drug delivery device 100 illustrated in FIGS. 1A-1B or the drug delivery device 200 illustrated in FIGS. 2A-2C). In the embodiment illustrated in FIG. 4, a method 300 of manufacturing a drug delivery device includes a task 310 of refining (e.g., purifying) one or more cannabinoids (e.g., Cannabidiol and/or Tetrandrocannabinol). The task 310 of refining the one or more cannabinoids may include extracting live resin from the cannabis sativa plant and/or the indica plant and refining the extracted resin into a crystalline form (i.e., crystalline resin). The method 300 may also include a task 320 of mixing the crystalline resin with a carrier oil (e.g., medium chain triglycerides (MCT oil)).

In one or more embodiments, the method 300 includes one or more tasks 330 of completing formation of the drug delivery device. In one or more embodiments, the task(s) 330 of completing formation of the drug delivery device includes inserting the mixture of the carrier oil and the one or more cannabinoids into a core, and forming a skin covering the core. In one or more embodiments, the task 330 of completing formation of the drug delivery device may also include shaping the core and the skin into the desired shape (e.g., an annular ring). In one or more embodiments, the shaping of the core and the skin may include co-extrusion of the core and the skin, cutting the extruded core and skin into segment or pieces having the desired length, and connecting ends of each individual segment together to form the ring-shaped drug delivery device. In one or more embodiments, the task 330 of completing formation of the drug delivery device may include forming an outer skin (e.g., a hollow tube) into a desired shape (e.g., ring) and connecting opposite ends of the outer skin together with a coupling member (e.g., a solid or hollow plug). The task 330 of completing formation of the drug delivery device may also include injecting (e.g., with a needle) the mixture of the carrier oil and the one or more cannabinoids into a reservoir defined in an interior of the outer skin.

While certain embodiments of the present invention have been illustrated and described, it is understood by those of ordinary skill in the art that certain modifications and changes can be made to the described embodiments without departing from the spirit and scope of the present invention as defined by the following claims, and equivalents thereof. As used herein, the term “substantially,” “about,” and similar terms are used as terms of approximation and not as terms of degree, and are intended to account for the inherent deviations in measured or calculated values that would be recognized by those of ordinary skill in the art. Additionally, as used herein, when a component is referred to as being “on” another component, it can be directly on the other component or components may also be present therebetween.

What is claimed is:

1. A drug delivery device configured to be inserted transvaginally, the drug delivery device comprising:
an outer skin defining a reservoir; and
a mixture of a carrier oil and one or more cannabinoids inside the reservoir,
wherein the outer skin is permeable to the mixture of the carrier oil and the one or more cannabinoids.

2. The drug delivery device of claim 1, wherein the drug delivery device is a resilient ring.

3. The drug delivery device of claim 1, wherein the one or more cannabinoids includes Cannabidiol and/or Tetrahydrocannabinol.

4. The drug delivery device of claim 1, wherein the one or cannabinoids includes less than approximately 0.03% Tetrahydrocannabinol.

5. The drug delivery device of claim 1, wherein the carrier oil comprises one or more medium chain triglycerides.

6. The drug delivery device of claim 1, wherein the outer skin is substantially free of cannabinoids prior to transvaginal insertion.

7. A method of treating symptoms associated with a menstrual cycle, the method comprising inserting the drug delivery device of claim 1 transvaginally into a user.

8. A method of manufacturing the drug delivery device of claim 1, the method comprising:
forming the outer skin;
shaping the outer skin into a desired shape of the drug delivery device;
refining the one or more cannabinoids into a crystalline resin;
mixing the crystalline resin with the carrier oil to form the mixture; and
injecting the mixture of the carrier oil and the one or more cannabinoids into the reservoir defined by the outer skin.

9. The method of claim 8, wherein the shaping the outer skin into the desired shape comprises:
extruding the outer skin to form an extruded outer skin;
cutting the extruded outer skin into at least one individual segment having a desired length; and
connecting ends of the at least one individual segment together to form the drug delivery device.

10. The method of claim 8, wherein the refining the one or more cannabinoids comprises:
extracting live resin from at least one of a cannabis sativa plant and an indica plant; and
refining the live resin extracted from the at least one of the cannabis sativa plant and the indica plant into the crystalline resin.
